(19) United States
(12) Patent Application Publication
(10) Pub. No.: US 2026/0074032 A1
(43) Pub. Date: Mar. 12, 2026

(54) APPARATUS AND METHOD FOR RECOMMENDING SIMILAR CLINICAL TRIAL DATA

(71) Applicant: MEDIAIPLUS, INC., Seongnam-si (KR)

(72) Inventors: Ji Hee JUNG, Seongnam-si Gyeonggi-do (KR); Yong Jang JO, Seongnam-si Gyeonggi-do (KR); Nam Goo SONG, Suwon-si Gyeonggi-do (KR)

(21) Appl. No.: 19/386,960

(22) Filed: Nov. 12, 2025

Related U.S. Application Data
(63) Continuation of application No. PCT/KR2024/097064, filed on Dec. 17, 2024.

(30) Foreign Application Priority Data
Apr. 29, 2024 (KR) 10-2024-0056811

(51) Int. Cl. G16H 10/20; G06F 40/268

(52) U.S. Cl. CPC G16H 10/20; G06F 40/268

(57) ABSTRACT

Disclosed are an apparatus and a method for recommending similar clinical trial data to extract clinical trial data similar to clinical trial data which is input by a user. A similar clinical trial data recommending apparatus according to an exemplary embodiment may include a preprocessor which classifies metadata and natural language data included in clinical trial data and generates a token for the natural language data; a feature extractor which generates an embedding vector based on the metadata and the token; and a data recommender which extracts one or more similar clinical trial data within a predetermined distance, among one or more previously stored clinical trial data, based on a distance between an embedding vector generated from input clinical trial data which is requested to be searched by a user and an embedding vector generated from one or more previously stored clinical trial data.

### CROSS-REFERENCE TO RELATED APPLICATIONS

This application is the National Stage filing under 35 U.S.C. 371 of International Application No. PCT/KR2024/097064, filed on December 27, 2024, which claims the benefit of K.R application No. 10-2024-0056811, filed on April 29, 2024, the contents of which are all hereby incorporated by reference herein in their entirety.

### TECHNICAL FIELD

The present disclosure relates to an apparatus and a method for recommending similar clinical trial data to extract clinical trial data similar to clinical trial data which is input by a user.

### BACKGROUND ART

Recently, in accordance with the global trend of opening clinical trial information to the public, there has been a growing interest in the utilization of clinical trial data. However, in the related art, the clinical trials have been managed through a paper-based management system (case report form, CRF) and have been statistically analyzed to verify hypotheses or objectives of the clinical trials.

Such paper-based clinical trial data management is extremely vulnerable in terms of data storage, maintenance, and security and has problems in that the data sharing, data reprocessing, variability or flexibility of testing or review periods, subsequent reference, and utilization are extremely restricted. In order to solve this problem, some electronic data-based clinical trial management systems (electronic case report form, eCRF) are being studied.

This invention was filed with support from the “2025 Global Startup Commercialization Support Program” funded by Gyeonggi Province and the Gyeonggi Business & Science Accelerator.

### SUMMARY

### Technical Problem

An object of the present disclosure is to provide an apparatus and a method for recommending similar clinical trial data to extract clinical trial data similar to clinical trial data which is input by a user.

### Technical Solution

According to another aspect, a similar clinical trial data recommending apparatus may include a preprocessor which classifies metadata and natural language data included in clinical trial data and generates a token for the natural language data; a feature extractor which generates an embedding vector based on the metadata and the token; and a data recommender of extracting one or more similar clinical trial data within a predetermined distance, among one or more previously stored clinical trial data, based on a distance between an embedding vector generated from input clinical trial data which is requested to be searched by a user and an embedding vector generated from one or more previously stored clinical trial data.

The preprocessor may generate a one-hot encoding vector for the metadata and generate a token from which at least one of special characters and stop words included in the natural language data is removed.

The feature extractor may include a first embedding model which generates an embedding vector for metadata based on the one-hot encoding vector; and a second embedding model which generates an embedding vector for natural language data based on the token.

The feature extractor may further include an ensemble model which receives the embedding vector output from the first embedding model and the embedding vector output from the second embedding model to generate an embedding vector for clinical trial data.

The feature extractor may generate a document term matrix for the token.

The second embedding model may receive a document term matrix to perform matrix factorization to generate a clinical trial data latent matrix and a term latent matrix.

The clinical trial data latent matrix may be configured by a matrix having a magnitude of “number of clinical trials × K” and the term latent matrix may be configured by a matrix having a magnitude of “K × number of terms”.

The data recommender may calculate a distance by determining each row which configures the clinical trial data latent matrix as an embedding vector of the clinical trial data.

The data recommender may calculate a distance between clinical trial data using a weighted sum of a distance based on an embedding vector output from the first embedding model and a distance based on an embedding vector output from the second embedding model.

According to another aspect, a similar clinical trial data recommending method which is carried out on a computing device including one or more processors and a memory which stores one or more programs executed by the one or more processors may include a preprocessing step of classifying metadata and natural language data included in clinical trial data and generates a token for the natural language data; a feature extracting step of generating an embedding vector based on the metadata and the token; and a data recommending step of extracting one or more similar clinical trial data within a predetermined distance, among one or more previously stored clinical trial data, based on a distance between an embedding vector generated from input clinical trial data which is requested to be searched by a user and an embedding vector generated from one or more previously stored clinical trial data.

In the preprocessing step, a one-hot encoding vector for the metadata may be generated and a token from which at least one of special characters and stop words included in the natural language data is removed may be generated.

The feature extracting step may include a first embedding model which generates an embedding vector for metadata based on the one-hot encoding vector; and a second embedding model which generates an embedding vector for natural language data based on the token.

The feature extracting step may further include an ensemble model which receives the embedding vector output from the first embedding model and the embedding vector output from the second embedding model to generate an embedding vector for clinical trial data.

In the feature extracting step, a document term matrix for the token may be generated.

The second embedding model may receive the document term matrix to perform matrix factorization to generate a clinical trial data latent matrix and a term latent matrix.

The clinical trial data latent matrix may be configured by a matrix having a magnitude of “number of clinical trials × K” and the term latent matrix may be configured by a matrix having a magnitude of “K × number of terms”.

In the data recommending step, a distance may be calculated by determining each row which configures the clinical trial data latent matrix as an embedding vector of the clinical trial data.

In the data recommending step, a distance between clinical trial data may be calculated using a weighted sum of a distance based on the embedding vector output from the first embedding model and a distance based on the embedding vector output from the second embedding model.

### Advantageous Effects

According to the present disclosure, clinical trial data similar to the clinical trial data which is input by the user may be quickly and effectively extracted to be provided to the user.

### BRIEF DESCRIPTION OF DRAWINGS

FIG. 1 is a diagram of a similar clinical trial data recommending apparatus according to an exemplary embodiment.

FIG. 2 is an exemplary view for explaining an environment where a similar clinical trial data recommending apparatus according to an exemplary embodiment operates.

FIG. 3 is a diagram of a feature extractor according to an exemplary embodiment.

FIG. 4 is a flowchart illustrating a similar clinical trial data recommending method according to an exemplary embodiment.

### DETAILED DESCRIPTIONS OF EXEMPLARY EMBODIMENTS

Hereinafter, an exemplary embodiment of the present disclosure will be described in detail with reference to the accompanying drawings. In the description of the present disclosure, a detailed description of known configurations or functions incorporated herein will be omitted when it is determined that the detailed description may make the subject matter of the present disclosure unclear. Further, the terms to be described below are defined considering the functions in the present disclosure and may vary depending on the intention or usual practice of a user or operator. Accordingly, the terms need to be defined based on details throughout this specification.

Hereinafter, exemplary embodiments of a similar clinical trial data recommending apparatus and method will be described in detail with reference to drawings.

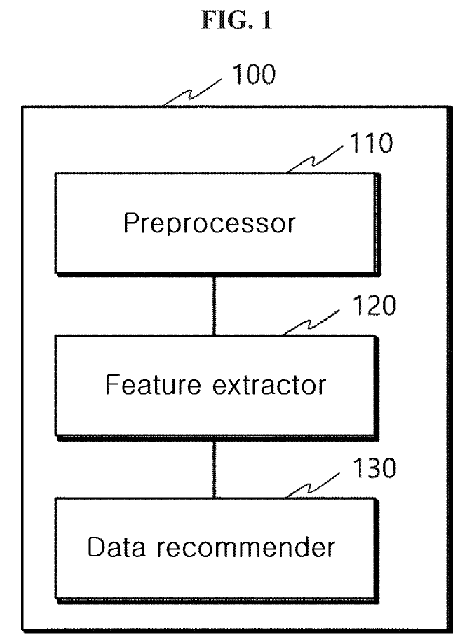

FIG. 1 is a diagram of a similar clinical trial data recommending apparatus according to an exemplary embodiment.

According to an exemplary embodiment, a similar clinical trial data recommending apparatus 100 may include a preprocessor 110, a feature extractor 120, and a data recommender 130.

### Mode for carrying out the invention

According to an exemplary embodiment, the preprocessor 110 may classify metadata and natural language data included in clinical trial data and generate a token for the natural language data.

For example, the preprocessor 110 may receive clinical trial data from the user. Further, the preprocessor 110 may collect clinical trial data from an external server device.

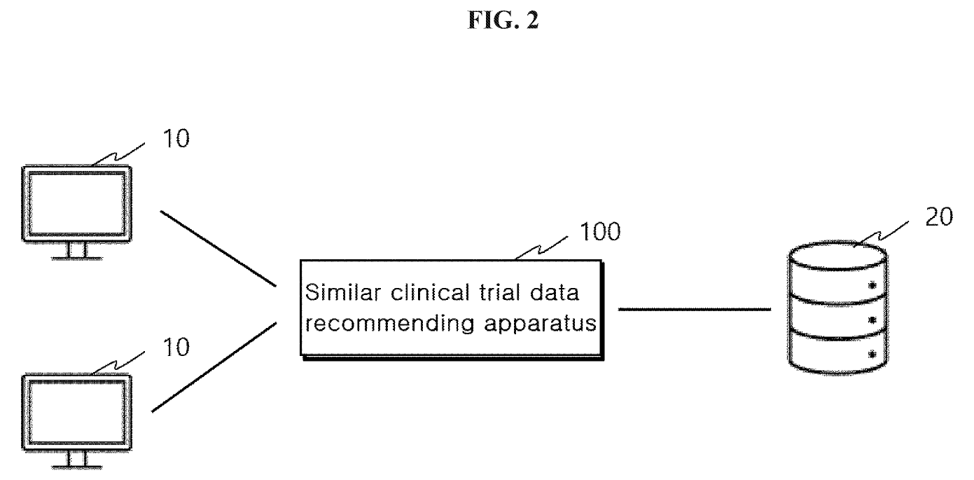

For example, as illustrated in FIG. 2, the similar clinical trial data recommending apparatus 110 may be connected to one or more user terminals 10 and an external server 20.

According to an example, the preprocessor 110 may classify metadata and natural language data from one or more clinical trial data received from the user terminal 10 or the external server 20. For example, the clinical trial data received from the user terminal may be a clinical trial keyword configured by at least one of a title, a clinical phase, intervention information about drugs or medical devices, a clinical location, indication, progress or recruitment status information, and patient eligibility criteria. For example, the user terminal 10 may be implemented by a smart phone, a tablet PC, a notebook, or a desktop.

For example, the metadata may be information about a CRIS registration number, an approval status, or an approved date. The natural language data may represent data configured by natural languages, such as a title, summary, and clinical trial results, rather than the metadata.

According to an example, the preprocessor 110 may generate a one-hot encoding vector for the metadata and generate a token from which special characters and stop words included in the natural language data are removed.

For example, the special characters and the stop words may be set in advance. The preprocessor 110 may tokenize after deleting the previously determined stop words from the clinical trial data or delete the stop words after tokenizing. For example, the stop words may include articles, prepositions, conjunctions, and interjections.

According to an example, the preprocessor 110 may calculate a term frequency. Next, the preprocessor 110 may generate a label based on a term and a frequency and then assign the label to the token. For example, a label configured by (frequency: 1000 times, term) may be assigned to each token.

According to an example, the preprocessor 110 may analyze a morpheme for each term and generate a pair of term and morpheme and then calculate a frequency. Next, the preprocessor 110 may generate a label based on a term-morpheme pair and a frequency and then assign the label to the token. For example, a label configured by (frequency: 1000 times, (term, morpheme)) may be assigned to each token.

According to the exemplary embodiment, the feature extractor 120 may generate an embedding vector based on the metadata and the token.

For example, the feature extractor 120 may include a first embedding model 121 which generates an embedding vector for metadata based on the one-hot encoding vector and a second embedding model 123 which generates an embedding vector for natural language data based on a token.

According to an example, the feature extractor 120 may transmit an embedding vector of the first embedding model 121 and an embedding vector of the second embedding model 123 to the data recommender 130 or generate one embedding vector to transmit the embedding vector to the data recommender 130.

According to an exemplary embodiment, the feature extractor 120 may further include an ensemble model 125 which receives the embedding vector output from the first embedding model and the embedding vector output from the second embedding model to generate an embedding vector for clinical trial data. The feature extractor 120 may generate one embedding vector for one clinical trial data through the ensemble model 125.

According to an exemplary embodiment, the feature extractor 120 may generate a document term matrix for the token. For example, the document term matrix may be configured by a clinical trial data axis and a term axis. That is, a magnitude of the document term matrix may be (number of clinical trials × K, ‘K × number of terms). At this time, K may be a hyper parameter representing a topic number. For example, in the document term matrix, the clinical trials and the terms may have a space of K. If K is set to be large, various information may be obtained and if K is set to be small, a noise other than key information may be removed.

For example, the document term matrix may be configured through a token assigned with a label configured by terms and a frequency or a token assigned with a label configured by a term-morpheme pair and a frequency. When the term-morpheme pair is used, in the magnitude of the document term matrix, the number of terms may be the number of term-morpheme pairs.

According to an exemplary embodiment, the second embedding model 123 may receive a document term matrix to perform matrix factorization to generate a clinical trial data latent matrix and a term latent matrix. For example, the matrix factorization may be non-negative matrix factorization.

The matrix which the magnitude of the row and the column is (number of clinical trials, number of terms) is classified into a clinical trial data latent matrix (first matrix) indicating embedding for clinical trials and a term latent matrix (second matrix) indicating embedding for terms and a process of obtaining two matrices may be configured by a method of updating a weight by means of non-negative matrix factorization.

According to an exemplary embodiment, the clinical trial data latent matrix may be configured by a matrix having a magnitude of “number of clinical trials × K” and the term latent matrix may be configured by a matrix having a magnitude of “K × number of terms”. For example, the clinical trial data latent matrix may be configured by a clinical trial data axis and a topic axis. The term latent matrix may be configured by a topic axis and a term axis.

For example, when the clinical trial data latent matrix may be configured by a matrix having a magnitude of “number of clinical trials × K”, the feature extractor 120 may generate as many embedding vectors as the number of clinical trials from the clinical trial data latent matrix. For example, an embedding vector corresponding to clinical trial data to which each row of the clinical trial data latent matrix is input may be output.

According to an exemplary embodiment, the data recommender 130 may extract one or more similar clinical trial data within a predetermined distance, among one or more previously stored clinical trial data, based on a distance between an embedding vector generated from input clinical trial data which is requested to be searched by a user and an embedding vector generated from one or more previously stored clinical trial data.

For example, the data recommender 130 may measure a distance of an embedding vector for clinical trial data input by the user and an embedding vector generated from the previously stored clinical trial data. At this time, the embedding vector generated from the previously stored clinical trial data may be stored in a vector database and the data recommender 130 may calculate a distance based on the embedding vector stored in the vector database.

According to an exemplary embodiment, the data recommender 130 may determine each row which configures the clinical trial data latent matrix as an embedding vector of the clinical trial data to calculate a distance. For example, a distance of each embedding vector corresponding to the clinical trial data generated from the clinical trial data latent matrix having a magnitude of “number of clinical trials × K” and an embedding vector for clinical trial data input by the user may be calculated. The data recommender 130 may determine previously stored clinical trial data having a distance between embedding vectors within a predetermined reference distance as similar clinical trial data.

According to an exemplary embodiment, the data recommender 130 may calculate a distance between clinical trial data using a weighted sum of a distance based on an embedding vector output from the first embedding model and a distance based on an embedding vector output from the second embedding model. For example, the feature extractor 120 may output an embedding vector for metadata and an embedding vector for natural language data. In this case, the data recommender 130 may calculate a distance of the embedding vector for the metadata and the embedding vector for the natural language data and assign a weight to the calculated distance to calculate a distance between the clinical trial data input by the user and the previously stored clinical trial data.

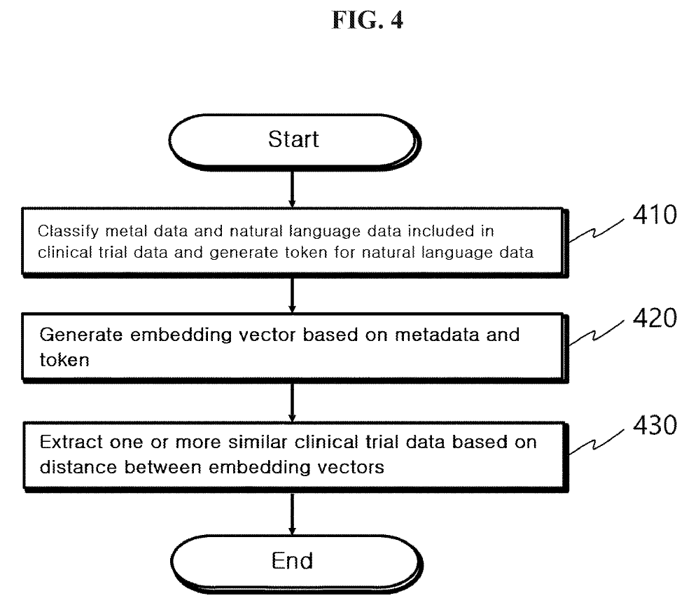

FIG. 4 is a flowchart illustrating a similar clinical trial data recommending method according to an exemplary embodiment.

According to an exemplary embodiment, the similar clinical trial data recommending apparatus may be a computing device including one or more processors and a memory which stores one or more programs executed by one or more processors.

According to an exemplary embodiment, the similar clinical trial data recommending apparatus may classify metadata and natural language data included in clinical trial data and generate a token for the natural language data in step 410 and generate an embedding vector based on the metadata and the token in step 420. Next, the similar clinical trial data recommending apparatus may extract one or more similar clinical trial data within a predetermined distance, among one or more previously stored clinical trial data, based on a distance between an embedding vector generated from input clinical trial data which is requested to be searched by a user and an embedding vector generated from one or more previously stored clinical trial data in step 430.

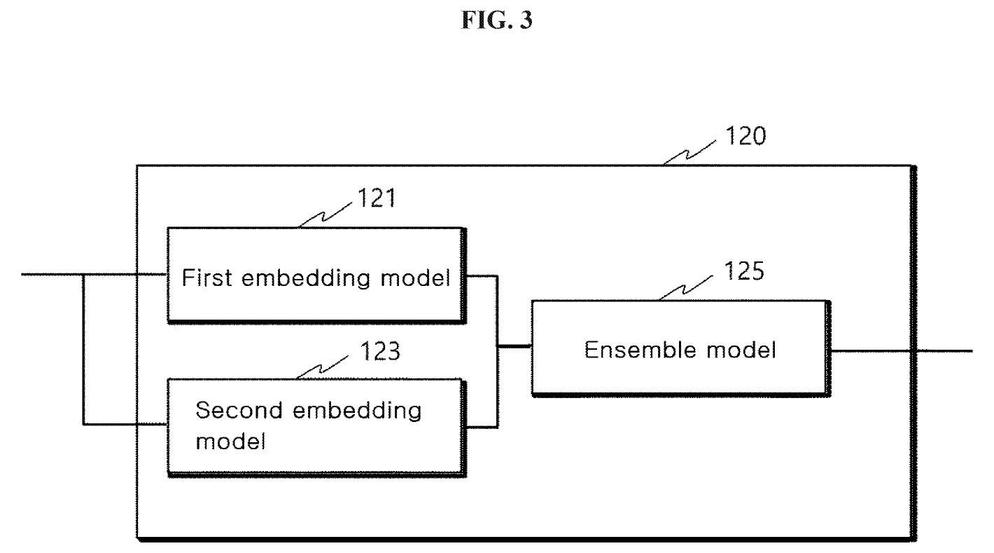

Among the exemplary embodiments of FIG. 4, exemplary embodiments that overlap with the contents described with reference to FIGS. 1 to 3 are omitted.

An aspect of the present disclosure may also be implemented as computer-readable codes written on a computer-readable recording medium. Codes and code segments which implement the program may be easily deduced by a computer programmer in the art. The computer readable recording medium may include all kinds of recording devices in which data, which are capable of being read by a computer system, are stored. Examples of the computer-readable recording media include ROM, RAM, CD-ROM, magnetic tape, floppy disk, optical disk and the like. Further, the computer readable recording medium is distributed in computer systems connected through a network to be written and executed with a computer readable code in a distributed manner.

For now, the present disclosure has been described with reference to the exemplary embodiments. It is understood to those skilled in the art that the present disclosure may be implemented as a modified form without departing from an essential characteristic of the present disclosure. Accordingly, the scope of the present disclosure is not limited to the above-described embodiment, but should be construed to include various embodiments within the scope equivalent to the description of the claims.

### Industrial Applicability

The present disclosure is applicable to the industry of clinical trials.

1. A similar clinical trial data recommending apparatus, comprising:
a preprocessor which classifies metadata and natural language data included in clinical trial data and generates a token for the natural language data;
a feature extractor which generates an embedding vector based on the metadata and the token; and
a data recommender which extracts one or more similar clinical trial data within a predetermined distance, among one or more previously stored clinical trial data, based on a distance between an embedding vector generated from input clinical trial data which is requested to be searched by a user and an embedding vector generated from the one or more previously stored clinical trial data.

2. The similar clinical trial data recommending apparatus according to claim 1, wherein the preprocessor generates a one-hot encoding vector for the metadata and generates the token from which at least one of special characters and stop words included in the natural language data is removed.

3. The similar clinical trial data recommending apparatus according to claim 2, wherein the feature extractor includes:
a first embedding model which generates an embedding vector for the metadata based on the one-hot encoding vector; and
a second embedding model which generates an embedding vector for the natural language data based on the token.

4. The similar clinical trial data recommending apparatus according to claim 3, wherein the feature extractor further includes an ensemble model which receives the embedding vector output from the first embedding model and the embedding vector output from the second embedding model to generate an embedding vector for the clinical trial data.

5. The similar clinical trial data recommending apparatus according to claim 3, wherein the feature extractor generates a document term matrix for the token.

6. The similar clinical trial data recommending apparatus according to claim 5, wherein the second embedding model receives the document term matrix to perform matrix factorization to generate a clinical trial data latent matrix and a term latent matrix.

7. The similar clinical trial data recommending apparatus according to claim 6, wherein the clinical trial data latent matrix is configured by a matrix having a magnitude of “number of clinical trials × K” and the term latent matrix is configured by a matrix having a magnitude of “K × number of terms”.

8. The similar clinical trial data recommending apparatus according to claim 7, wherein the data recommender calculates a distance by determining each row which configures the clinical trial data latent matrix as an embedding vector of the clinical trial data.

9. The similar clinical trial data recommending apparatus according to claim 3, wherein the data recommender calculates a distance between the clinical trial data using a weighted sum of a distance based on the embedding vector output from the first embedding model and a distance based on the embedding vector output from the second embedding model.

10. A similar clinical trial data recommending method which is carried out on a computing device including one or more processors and a memory which stores one or more programs executed by the one or more processors, the method comprising:
a preprocessing step of classifying metadata and natural language data included in clinical trial data and generates a token for the natural language data;
a feature extracting step of generating an embedding vector based on the metadata and the token; and
a data recommending step of extracting one or more similar clinical trial data within a predetermined distance, among one or more previously stored clinical trial data, based on a distance between an embedding vector generated from input clinical trial data which is requested to be searched by a user and an embedding vector generated from the one or more previously stored clinical trial data.

11. The similar clinical trial data recommending method according to claim 10, wherein in the preprocessing step, a one-hot encoding vector for the metadata is generated and the token from which at least one of special characters and stop words included in the natural language data is removed is generated.

12. The similar clinical trial data recommending method according to claim 11, wherein the feature extracting step includes:
a first embedding model which generates an embedding vector for the metadata based on the one-hot encoding vector; and
a second embedding model which generates an embedding vector for the natural language data based on the token.

13. The similar clinical trial data recommending method according to claim 12, wherein the feature extracting step further includes an ensemble model which receives the embedding vector output from the first embedding model and the embedding vector output from the second embedding model to generate an embedding vector for the clinical trial data.

14. The similar clinical trial data recommending method according to claim 12, wherein in the feature extracting step, a document term matrix for the token is generated.

15. The similar clinical trial data recommending method according to claim 14, wherein the second embedding model receives the document term matrix to perform matrix factorization to generate a clinical trial data latent matrix and a term latent matrix.

16. The similar clinical trial data recommending method according to claim 15, wherein the clinical trial data latent matrix is configured by a matrix having a magnitude of “number of clinical trials × K” and the term latent matrix is configured by a matrix having a magnitude of “K × number of terms”.

17. The similar clinical trial data recommending method according to claim 16, wherein in the data recommending step, a distance is calculated by determining each row which configures the clinical trial data latent matrix as an embedding vector of the clinical trial data.

18. The similar clinical trial data recommending method according to claim 12, wherein in the data recommending step, a distance between the clinical trial data is calculated using a weighted sum of a distance based on the embedding vector output from the first embedding model and a distance based on the embedding vector output from the second embedding model.
